(19) United States
(12) Patent Application Publication
(10) Pub. No.: US 2008/0262426 A1
(43) Pub. Date: Oct. 23, 2008

(54) Controller

(75) Inventors: Takashi Hachiya, Tokyo (JP); Kazumasa Masuda, Tokyo (JP)

Correspondence Address: KNOBBE MARTENS OLSON & BEAR LLP, 2040 MAIN STREET, FOURTEENTH FLOOR, IRVINE, CA, 92614, US

(73) Assignee: Nemoto Kyorindo Co., Ltd, Tokyo (JP)

(21) Appl. No.: 11/570,229

(22) PCT Filed: Jun. 10, 2005
(86) PCT No.: PCT/JP2005/010655

§ 371 Date: Dec. 7, 2006

(30) Foreign Application Priority Data
May 10, 2004 (JP) 2004172514

(51) Int. Cl. A61M 5/172

(52) U.S. Cl. 604/131

(57) ABSTRACT

A controller which can be kept clean easily and has no influence on magnetism is provided.

A light ray from light-emitting element 401 mounted on body 101 of a movable apparatus is detected by light-receiving element 402 mounted on body 101 via first optical fiber 431, wavelength-variable mechanism 421 of controller body 420 and second optical fiber 432. Wavelength-variable mechanism 421 changes a wavelength of the light ray in accordance with manual operation. The wavelength is detected by light-receiving element 402, and an operation of a movable mechanism is controlled. Since controller body 420 is not connected to body 101 via a wire, the controller has no influence on magnetism. Since an electronic circuit is not mounted on controller body 420, for example, controller body 420 can be disinfected with disinfectant or fumigation and also can be formed as a disposable device.

### TECHNICAL FIELD

The present invention relates to a controller for controlling the operation of a movable apparatus including a movable mechanism mounted on an apparatus body, and more particularly, to a controller including a controller unit removably mounted on the apparatus body.

### BACKGROUND ART

Presently available diagnostic imaging apparatuses for capturing diagnostic images of patients include CT (Computed Tomography) scanners, MRI (Magnetic Resonance Imaging) apparatuses, PET (Positron Emission Tomography) apparatuses, SPECT (Single Photon Emission Computed Tomography) apparatuses, ultrasonic diagnostic apparatuses and the like. Angiography apparatuses, MBA (MR angiography) apparatuses and the like are currently used as diagnostic imaging apparatuses for capturing vascular images of patients.

When the abovementioned diagnostic imaging apparatuses are used, a liquid such as a contrast medium or physiological saline may be injected into a patient. Chemical liquid injectors for automatically performing the injection have been put into practical use. A liquid syringe, for example including a cylinder member and a piston member slidably inserted into the cylinder member, is mounted on such a chemical liquid injector. A syringe driving mechanism presses the piston member into the cylinder member.

The cylinder member is filled with a liquid and connected to a blood vessel of a human body near the surface thereof through an extension tube and an injection needle. Thus, the liquid in the liquid syringe is injected with pressure into the blood vessel of the human by the chemical liquid injector. Some of the chemical liquid injectors can automatically perform the injection in accordance with initial settings, and others can perform the injection based on real-time control. Some of the chemical liquid injectors performing the injection based on real-time control include a controller integral with the body of the injector, and others have a separately formed controller body of a controller from the body of the injector.

In such a case, for example, a manual operation member is slidably mounted on the controller body which contains a signal producing circuit connected to the manual operation member. The signal producing circuit comprises a variable resistor, for example, and produces a driving control signal in response to slide operation of the manual operation member.

The controller body is connected to one end of a flexible wire cable having a sheathed conductor, and the other end of the wire cable is connected to the body of the chemical liquid injector. The body includes a driving circuit for driving a slider mechanism. The driving circuit is connected to the signal producing circuit of the controller unit through the conductor of the wire cable.

In the chemical liquid injector described above, when an operator holds the controller body and slides the manual operation member, the signal producing circuit produces a driving control signal in response thereto and supplies the signal to the driving circuit in the body of the chemical liquid injector through the wire cable. Since the driving circuit controls the operation of the slider mechanism in response to the supplied driving control signal the piston member of the liquid syringe is slid in accordance with the operation of the manual operation member of the controller.

In the abovementioned chemical liquid injector, the body of the chemical liquid injector and the controller formed as separate components are connected to each other through the flexible wire cable, and the injection operation in the body of the chemical liquid injector can be manually operated by the controller body, so that excellent usability is achieved. Chemical liquid injectors of the type having the abovementioned controller have been commercially manufactured and disclosed on the Internet and the like (see, for example, non-patent documents 1 to 4 below).

Non-patent document 1: “Dual Shot in product guides of Nemoto Kyorindo Co., Ltd” (retrieved in Feb. 13, 2004) (URL:http://www.nemoto-do.co.jp/seihin_ct.html#dual);

Non-patent document 2: “Auto Enhance A-60 in product guides of Nemoto Kyorindo Co., Ltd” (retrieved in Feb. 13, 2004) (URL:http://www.nemoto-do.co.jp/seihin_ct.html#a60);

Non-patent document 3: “Auto Enhance A-25 in product guides of Nemoto Kyorindo Co., Ltd” (retrieved in Feb. 13, 2004) (URL:http://www.nemoto-do.co.jp/seihin_ct.html#a25);

Non-patent document 4: “Sonic Shot 50 in product guides of Nemoto Kyorindo Co., Ltd” (retrieved in Feb. 13, 2004) (URL:http://www.nemoto-do.co.jp/seihin ang.mr.html.#sonic50)

### DISCLOSURE OF THE INVENTION

### Subject to be Solved by the Invention

In medical facilities where the abovementioned chemical liquid injector is used, operator's hands and fingers for manipulating the chemical liquid injector should be always kept clean. To this end, at least the controller body should be disinfected. However, it is difficult to disinfect the controller body containing the signal producing circuit and the like with disinfectant or fumigation.

If a controller body is formed of a controller unit removably mounted on the body of the chemical liquid injector and the controller unit is disposable, the controller unit can be always kept clean. However, the controller unit having the wire cable, the signal producing circuit and the like is not inexpensive, and in reality, it is difficult to form the controller unit as a disposable device.

Diagnostic imaging apparatuses used together with the above-mentioned chemical liquid injector include MRI apparatuses and MRA apparatuses which use magnetism. If the controller including the controller body containing various circuits connected to the body of the chemical liquid injector through the wire cable formed of the conductor is located close to the diagnostic imaging apparatus, the controller impairs the magnetic field of the diagnostic imaging apparatus.

The present invention has been made in view of the above-mentioned problems, and it is an object thereof to provide a controller which can be always kept clean easily and can be used close to a diagnostic imaging apparatus using magnetism.

### Means to Solve the Subject

The present invention provides a controller for controlling the operation of a movable mechanism mounted on a body of a movable apparatus. A controller according to a first aspect includes a controller body, an optical fiber, a wavelength-variable mechanism, a light-receiving element, and a driving control means.

The controller body is formed separately from the body of the apparatus. The optical fiber is flexible and has one end attached to the controller body and the other end attached to the body of the apparatus. The light-emitting element is mounted on the controller body and emits a light ray which enters the one end of the optical fiber. The wavelength-variable mechanism is mounted on the controller body and changes the wavelength of the light ray emitted by the light-emitting element and entering the one end of the optical fiber in accordance with manual operation. The light-receiving element is mounted on the body of the apparatus and detects the wavelength of the light ray emitted from the other end of the optical fiber. The driving control means is mounted on the body of the apparatus and controls the operation of the movable mechanism in accordance with the detection result of the light-receiving element.

A controller according to a second aspect of the present invention including a controller body, a first optical fiber, a light-emitting element, a second optical fiber, a wavelength-variable mechanism, a light-receiving element, and a driving control means. The first optical fiber is flexible and has one end attached to the body of the apparatus and the other end attached to the controller body. The light-emitting element is mounted on the body of the apparatus and emits a light ray which enters the one end of the first optical fiber. The second optical fiber is flexible and has one end attached to the controller body, the light ray emitted from the other end of the first optical fiber entering the one end, and the other end attached to the body of the apparatus. The wavelength-variable mechanism is mounted on the controller body and changes the wavelength of the light ray emitted from the other end of the first optical fiber and entering the one end of the second optical fiber in accordance with manual operation. The light-receiving element is mounted on the body of the apparatus and detects the wavelength of the light ray emitted from the other end of the second optical fiber.

Thus, in the first/second controllers of the present invention, when the wavelength-variable mechanism of the controller body connected to the body of the apparatus through the optical fiber is manually operated, the wavelength of the light ray detected by the light-receiving element of the body of the movable apparatus is changed to produce a driving control signal. As a result, the operation of the movable mechanism of the body of the apparatus is controlled in accordance with the manual operation of the controller body. In addition, in the first/second controllers of the present invention, the controller body and the body of the apparatus are not connected to each other through a wire cable formed of conductor, and in the second controller, the controller body does not include various circuits or the like which have complicated structures and are expensive.

Various means referred to in the present invention may be arranged to perform their functions, and may comprise dedicated hardware for performing a predetermined function, a data processing apparatus whose predetermined function is given by a computer program, a predetermined function performed in a data processing apparatus according to a computer program, or a combination thereof.

Various components referred to in the present invention do not need to be a separate entity. A plurality of means may be constructed as one member, a certain means may be part of another means, or a certain means may have a portion overlapping a portion of another means.

### EFFECT OF THE INVENTION

In the controller of the present invention, the operation of the movable mechanism of the body of the apparatus can be controlled in accordance with the manual operation of the controller body. In the first/second controllers of the present invention, since the controller body and the body of the apparatus are not connected through a wire cable formed of conductor, they can be used near the diagnostic imaging apparatus utilizing magnetism. In the second controller, since the controller body does not include various circuits or the like which have complicated structures and are expensive, the controller body can be disinfected with disinfectant or fumigation, or can be disposable.

### BRIEF DESCRIPTION OF THE DRAWINGS

FIG. 1 is a cross-sectional view showing the inner structure of a controller according to an embodiment of the present invention.

FIG. 2 is a perspective view showing a cross-sectional view showing a controller unit of the controller removed from an injection control unit corresponding to a body of an apparatus.

FIG. 3 is a perspective view showing the outer appearance of a chemical liquid injector.

FIG. 4 is a perspective view showing how to mount a liquid syringe on an injection head of the chemical liquid injector.

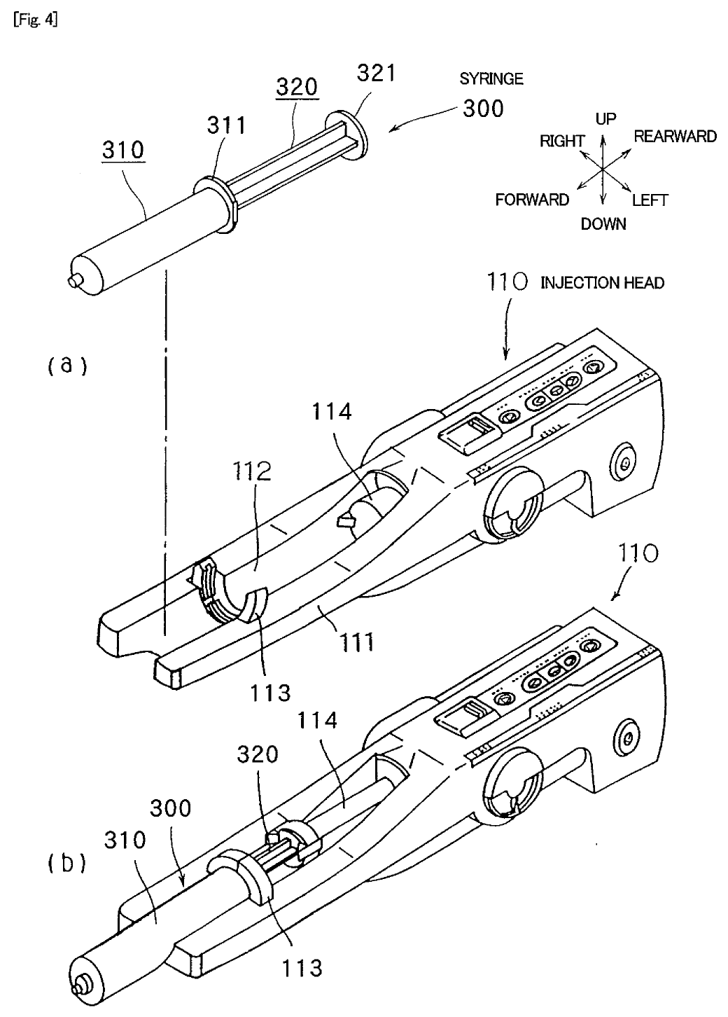

FIG. 5 is a perspective view showing the outer appearance of a diagnostic imaging system.

FIG. 6 is a block diagram showing the circuit structure of the diagnostic imaging system.

FIG. 7 is an exploded perspective view showing the inner structure of a controller unit in a modification.

FIG. 8 is an exploded perspective view showing the inner structure of a controller unit in a modification.

FIG. 9 is an exploded perspective view showing the inner structure of a controller unit in a modification.

### DESCRIPTION OF REFERENCE NUMERALS

100 CHEMICAL LIQUID INJECTOR
101 INJECTION CONTROL UNIT corresponding to body of apparatus
114 SYRINGE DRIVING MECHANISM serving as movable mechanism
130 COMPUTER BLOCK serving as driving control means
300 LIQUID SYRINGE
310 CYLINDER MEMBER
320 PISTON MEMBER
400 CONTROLLER
401 LIGHT-EMITTING ELEMENT
402 LIGHT-RECEIVING ELEMENT
410, 500 CONTROLLER UNIT
420, 502 CONTROLLER BODY
421 LIGHT-TRANSMITTING PLATE serving as wavelength-variable element
422, 513 MANUAL OPERATION MEMBER
431 FIRST OPTICAL FIBER
432 SECOND OPTICAL FIBER
520 SHUTTER MEMBER serving as optical path open/close mechanism

### BEST MODE FOR CARRYING THE INVENTION

### Configuration of Embodiment

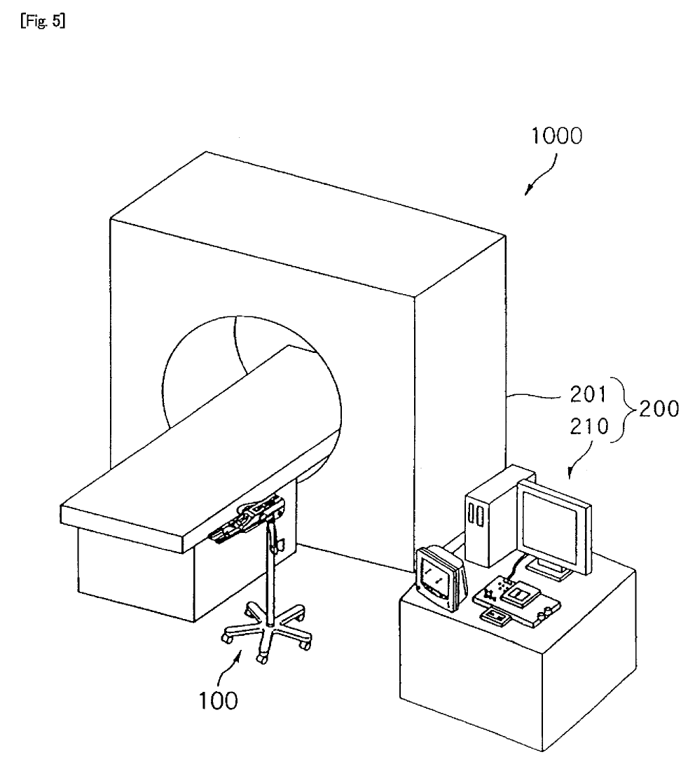

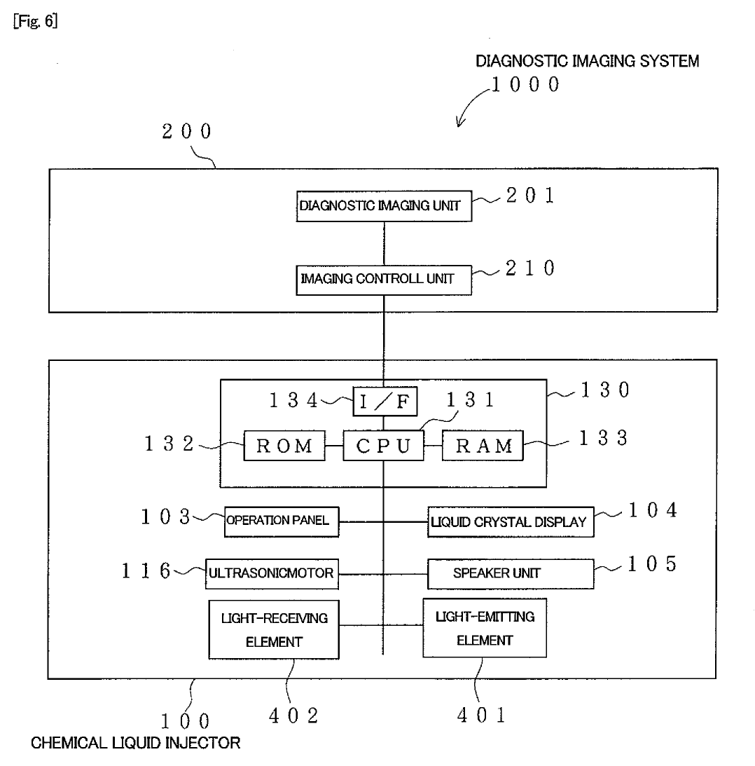

An embodiment of the present invention will hereinafter be described with reference to drawings. As shown in FIGS. 5 and 6, diagnostic imaging system 1000 of the embodiment has chemical liquid injector 100 serving as a movable apparatus and MRI apparatus 200 serving as a diagnostic imaging apparatus. Chemical liquid injector 100 and MRI apparatus 200 are wire-connected.

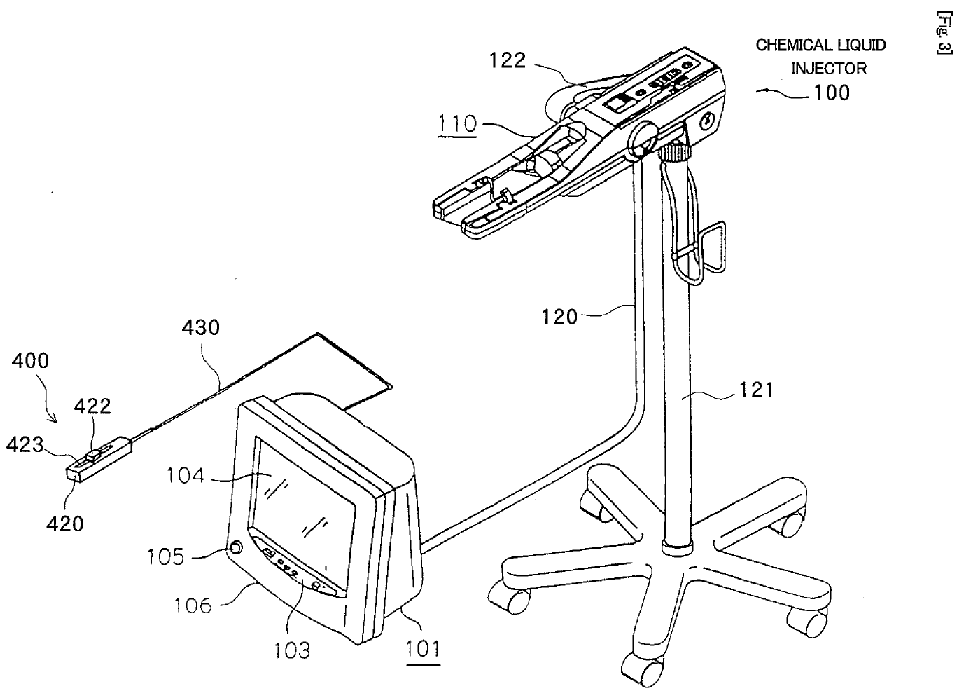

As shown in FIG. 3, chemical liquid injector 100 includes injection control unit 101 corresponding to a body of apparatus and injection head 110 constructed as separate components. Injection control unit 101 and injection head 110 are wire-connected through communication cable 120. Injection head 110 is attached to the top end of caster stand 121 by movable arm 122. Head body 111 of injection head 110 has concave portion 112 in the upper surface.

Liquid syringe 300 comprises cylinder member 310 and piston member 320 wherein piston member 320 is slidably inserted into cylinder member 310. Cylinder member 310 and piston member 320 have cylinder flange 311 and piston flange 321 formed in the outer circumferences of the trailing ends thereof, respectively. Cylinder member 310 has conduit 312 formed at the closed leading end.

In diagnostic imaging system 1000 of the embodiment, liquid syringe 300 is filled with a contrast medium as a liquid suitable for MRI apparatus 200. When chemical liquid injector 100 injects the contrast medium into a patient from liquid syringe 300, MRI apparatus 200 captures diagnostic images of the patient.

In chemical liquid injector 100 of the embodiment, syringe holding mechanism 113 which is openable and closable is formed in the forward section of concave portion 112 of injection head 110. Syringe holding mechanism 113 removably holds cylinder flange 311 of liquid syringe 300. Syringe driving mechanism 114 serving as a movable mechanism is disposed in the rearward section of concave portion 112 of injection head 110. Syringe driving mechanism 114 holds and slides piston member 320 of liquid syringe 300 held in concave portion 112. Syringe driving mechanism 114 has ultrasonic motor 116 as a driving source and slides piston member 320 with a screw mechanism (not shown) or the like.

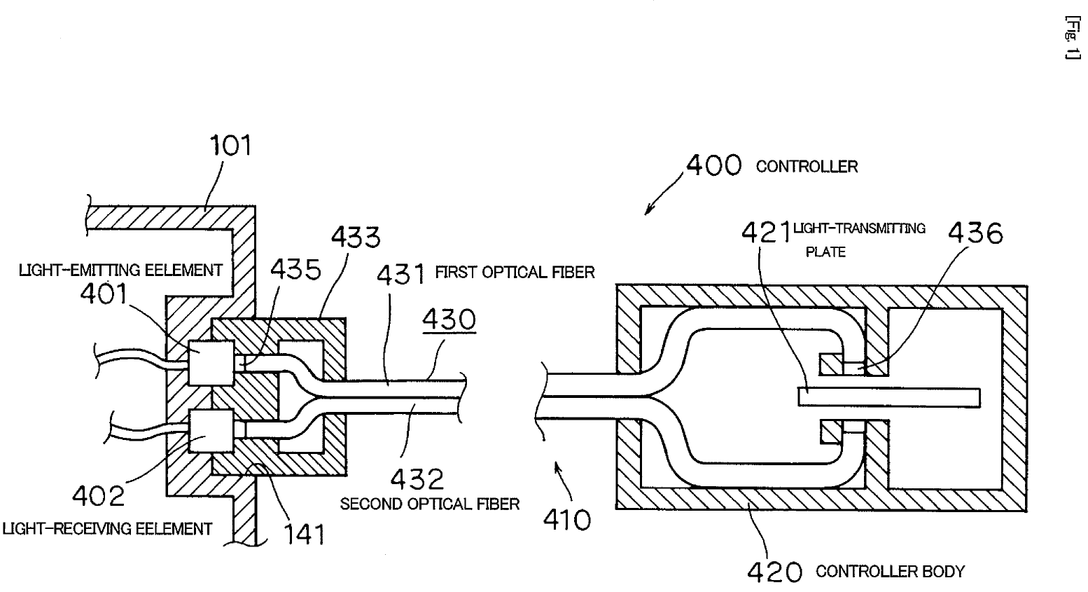

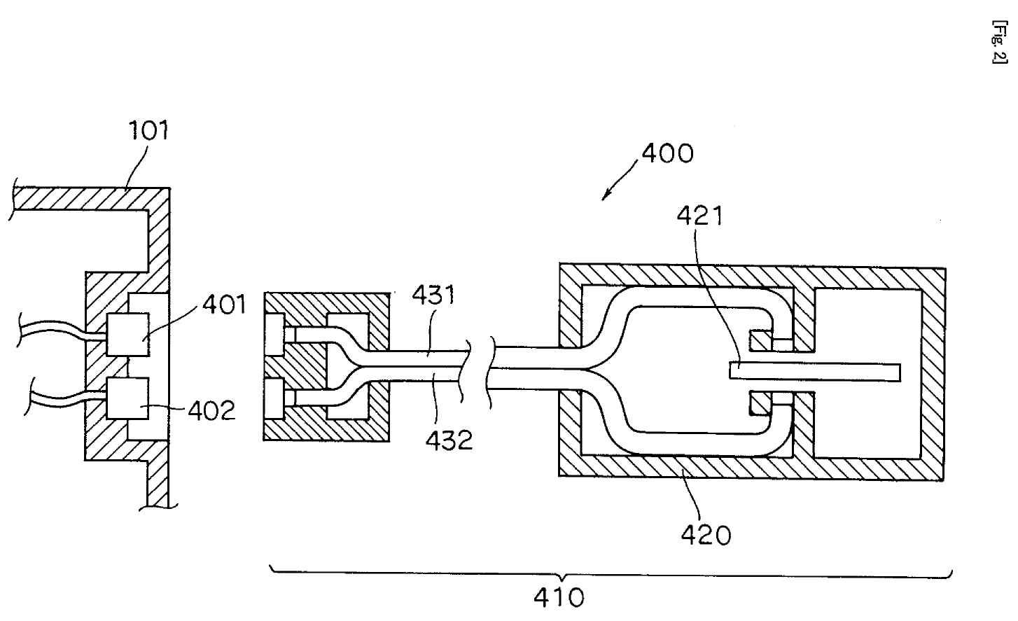

On the other hand, as shown in FIG. 3, injection control unit 101 has operation panel 103, liquid crystal display 104, speaker unit 105 and the like, all of which are disposed on the outer face of unit body 106. As shown in FIGS. 1 and 2, controller unit 400 is integrally formed with injection control unit 101.

As shown in FIG. 6, injection control unit 101 contains computer block 130 serving as a driving control means for controller 400. Computer block 130 is connected to respective portions such as operation panel 103, liquid crystal display 104, speaker unit 105, ultrasonic motor 116, light-emitting element 401, and light-receiving element 402.

Computer block 130 is formed of a so-called one-chip microcomputer provided with hardware such as CPU (Central Processing Unit) 131, ROM (Read Only Memory) 132, RAM (Random Access Memory) 133, communication I/F (Interface) 134 and the like.

A computer program is installed in ROM 132. CPU 131 performs various types of processing in accordance with the computer program to integrate and control the respective portions of chemical liquid injector 100. Although respective portions such as ultrasonic motor 116 and speaker unit 106 are actually connected to computer block 130 via a driving circuit and the like, direct connections are shown here to simplify the description.

Controller 400 has light-emitting element 401, light-receiving element 402, and controller unit 410. Controller unit 410 has controller body 420 and fiber cable 430 as main components. Injection control unit 101 has optical socket 141 as a concave portion having a predetermined shape formed in its outer face. Light-emitting element 401 and light-receiving element 402 are placed in optical socket 141.

Light-emitting element 401 comprises an electric bulb (not shown) combined with a collimator optical system, for example, and emits collimated beam of light in a predetermined wavelength band. Light-receiving element 402 comprises, for example, three photodiodes (not shown) correspond to color filters for RGB (Red, Green, and Blue). From the intensity of the received light ray detected by each of them, the wavelength thereof is detected by computer block 130.

Fiber cable 430 of controller unit 410 comprises first optical fiber 431 and second optical fiber 432 connected in parallel. Optical plug 433 is attached integrally with the trailing end of fiber cable 430. Optical plug 433 is removably attached to optical socket 141. When optical plug 433 is inserted into optical socket 141, the end of first optical fiber 431 is opposed and optically coupled to light-emitting element 401, and the end of second optical fiber 432 is opposed and optically coupled to light-receiving element 402.

Controller body 420 is formed to have an elongated box shape and is attached to the leading end of fiber cable 430. Within controller body 420, the ends of first and second optical fibers 431 and 432 are opposed to each other. The trailing ends of first and second optical fibers 431 and 432 are attached integrally to coupling optical systems 435 for realizing the optical coupling between light-emitting elements 401 and light-receiving element 402. The leading ends thereof are attached integrally to coupling optical systems 436 for realizing the optical coupling between them.

Elongated light-transmitting plate 421 serving as a wavelength-variable element is mounted on controller body 420 and is placed slidably in the longitudinal direction in the space between the opposite leading ends of first and second optical fibers 431 and 432. Light-transmitting plate 421 is formed of, for example, color filters for RGB arranged longitudinally in order and transmits light rays in different wavelengths in its different portions.

As shown in FIG. 3, manual operation member 422 is placed on the outer face of controller body 420 and is connected to light-transmitting plate 421 via a slit-shaped through-hole 423. When manual operation member 422 of controller body 420 is manually slid, light-transmitting plate 421 is slid accordingly to change the wavelength of the light ray emitted from first optical fiber 431 and entering second optical fiber 432.

The respective portions of controller unit 410 are made of material such as engineering plastics or the like which provides sufficient corrosion resistance, heat resistance, and strength, and which is not affected by magnetism.

### OPERATION OF THE EMBODIMENT

The operation of diagnostic imaging system 1000 of the embodiment in the abovementioned structure will be described in order. First, as shown in FIG. 5, injection head 110 of chemical liquid injector 100 is disposed near diagnostic imaging unit 201 of MRI apparatus 200, and liquid syringe 300 filled with a liquid such as a contrast medium is prepared for use together with an extension tube (not shown) and the like.

Then, liquid syringe 300 is connected to a patient (not shown) in diagnostic imaging unit 201 via the extension tube or the like. Liquid syringe 300 is mounted on injection head 110 of chemical liquid injector 100. In this state, when an operator makes entry to start operation on operation panel 103 of injection control unit 101 or the like, computer block 130 detects the operation and drives light-emitting element 401.

Light-emitting element 401 emits a light ray which then enters the trailing end of first optical fiber 431 of controller unit 410 and comes out of the leading end thereof. The light ray passes through light-transmitting plate 421, which can change the wavelength thereof. The light ray with the wavelength changed by light-transmitting plate 421 enters the leading end of second optical fiber 432 and comes out of the trailing end thereof. The light ray coming out of the trailing end is detected by light-receiving element 402, and the wavelength of the detected light ray is determined by computer block 130.

In the initial state, since manual operation member 422 is placed close to the trailing end of through-hole 423 of controller body 420 by an operator (not shown), the light ray passes through the portion of the color B in RGB light-transmitting plate 421, and the B color is detected by computer block 130. In response to the detection result, computer block 130 controls ultrasonic motor 116 to disable the operation thereof, so that syringe driving mechanism 114 is not driven unnecessarily in the initial state.

After that state, the operator slides manual operation member 422 to near the center of through-hole 423 of controller body 420. Then, the light ray passes through the portion of the color G in light-transmitting plate 421, and the G color is detected by computer block 130. In response to the detection result, computer block 130 operates syringe driving mechanism 114 at low speed to inject the liquid from liquid syringe 300 into the patient at low speed.

After that state, the operator slides manual operation member 422 to near the leading end of through-hole 423 of controller body 420. Then, the light ray passes through the portion of the color R in light-transmitting plate 421, and the R color is detected by computer block 130. In response to the detection result, computer block 130 operates syringe driving mechanism 114 at high speed to inject the liquid from liquid syringe 300 into the patient at high speed.

### EFFECT OF THE EMBODIMENT

In chemical liquid injector 100 of diagnostic imaging system 1000 of the embodiment, manual operation member 422 of controller body 420 can be manually operated to remotely control the liquid injection with syringe driving mechanism 114 of injection head 110 as described above.

In chemical liquid injector 100 of the embodiment, controller unit 410 does not include light-emitting element 401, light-receiving element 402, or the wires, and thus has no influence on a magnetic field. Thus, chemical liquid injector 100 can be easily used near MRI apparatus 200.

In chemical liquid injector 100 of the embodiment, controller unit 410 operated manually is removably mounted on injection control unit 101, and controller unit 410 does not include light-emitting element 401, light-receiving element 402, or the wires. Thus, controller unit 410 can be removed from injection control unit 101 and disinfected with disinfectant or boiling. As a result, controller unit 410 operated manually can be always kept clean.

In addition, since controller unit 410 which does not include light-emitting element 401, light-receiving element 402, or the wires is significantly inexpensive, it can be disposable and easily discarded after it is used once or several times. As a result controller unit 410 can be kept clean more reliably.

### MODIFICATIONS OF THE EMBODIMENT

The present invention is not in any way limited to the above-mentioned embodiment, but various changes and modifications may be made therein without departing from the scope of the invention. For example, in the above embodiment, chemical liquid injector 100 is shown as an example of the movable apparatus on which controller unit 410 is mounted to control the operation thereof. However, the present invention is applicable to various types of movable apparatuses.

In the above embodiment, light-transmitting plate 421 is clearly sectioned for a plurality of colors, and the operation of syringe driving mechanism 114 is controlled in a plurality of steps based on the colors. For example, light-transmitting plate 421 may be colored steplessly and the operation of syringe driving mechanism 114 may be controlled steplessly based on the color.

In the above embodiment, light-transmitting plate 421 is moved in association with the manual operation of manual operation member 422. For example, light-transmitting plate 421 may be fixed and the ends of first and second optical fibers 431 and 432 may be moved. It is possible to move the end of one of first and second optical fibers 431 and 432.

In the above embodiment, elongated light-transmitting plate 421 is supported slidably in the longitudinal direction in controller body 420 and slid together with manual operation member 422. For example, a disc light-transmitting plate may be rotatably supported and rotated together with the manual operation member (not shown).

In the above embodiment, light-transmitting plate 421 is used as an example of the wavelength-variable element for changing the wavelength of the light ray. As such a wavelength-variable element, for example, it is possible to use a reflective plate which reflects light rays with different wavelengths in different portions or a prism which transmits or reflects light rays with different wavelengths in different portions (not shown).

In the above embodiment, light-transmitting plate 421 and manual operation member 422 are simply mounted slidably on controller body 420. For example, it is possible to form an automatic returning mechanism formed of a coil spring or the like for automatically returning light-transmitting plate 421 and manual operation member 422 to the initial positions. Light-transmitting plate 421 and manual operation member 422 biased in this manner may be releasably held in an operation state by an operation holding mechanism of a predetermined structure (not shown).

In this case, releasing the operation holding mechanism causes the automatic returning mechanism to automatically return light-transmitting plate 421 and manual operation member 422 to return to the initial positions. Thus, the reset to the initial states can be performed easily and reliably. Since light-transmitting plate 421 and manual operation member 422 manually operated to the desired positions can be held by the operation holding mechanism, the operation state can be maintained as desired.

In the above embodiment, both of light-emitting and light-receiving elements 401 and 402 are mounted on injection control unit 101 and none of them are included in controller unit 410. For example, light-emitting element 401 may be mounted on controller body 420 (not shown). In this case, a power source such as a battery needs to be provided for controller body 420, but controller body 420 and injection control unit 101 do not need to be connected through wires or the like, thereby making it possible to minimize the influence on a magnetic field.

In the above embodiment, only one liquid syringe 200 is mounted on injection head 110 and only one manual operation member 422 is included in controller body 420. It is possible that a plurality of liquid syringes 200 are mounted on the injection head and a plurality of controller bodies 420 are provided for controller body 420 up to the same number as that of liquid syringes 300.

For example, in some chemical liquid injectors (not shown) actually used in MRI apparatus 200, a liquid syringe for a contrast medium and a liquid syringe for physiological saline are placed in parallel on an injection head. In such a case, two manual operation members 422 may be mounted on one controller body 420 to remotely control the two liquid syringes on the injection head individually.

In the above embodiment, the operation speed of syringe driving mechanism 114 on injection head 110 corresponds to the position of slid manual operation member 422 of controller unit 410. For example, the position of slid syringe driving mechanism 114 on injection head 110 may be matched with the position of slid manual operation member 422 of controller unit 410.

In the above embodiment, first and second optical fibers 431 and 432 are perpendicularly bent and opposed to each other within controller body 420. For example, an optical element (not shown) for reflecting a light ray perpendicularly may be used to form an optical path without bending first and second optical fibers 431 and 432 within controller body 420.

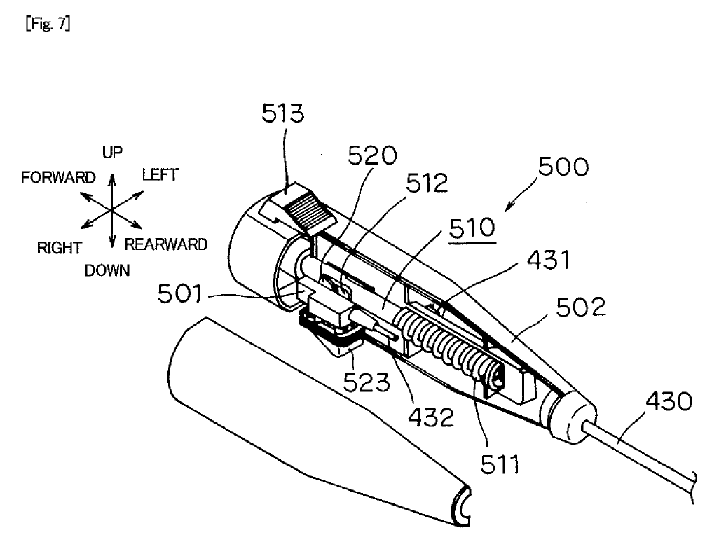

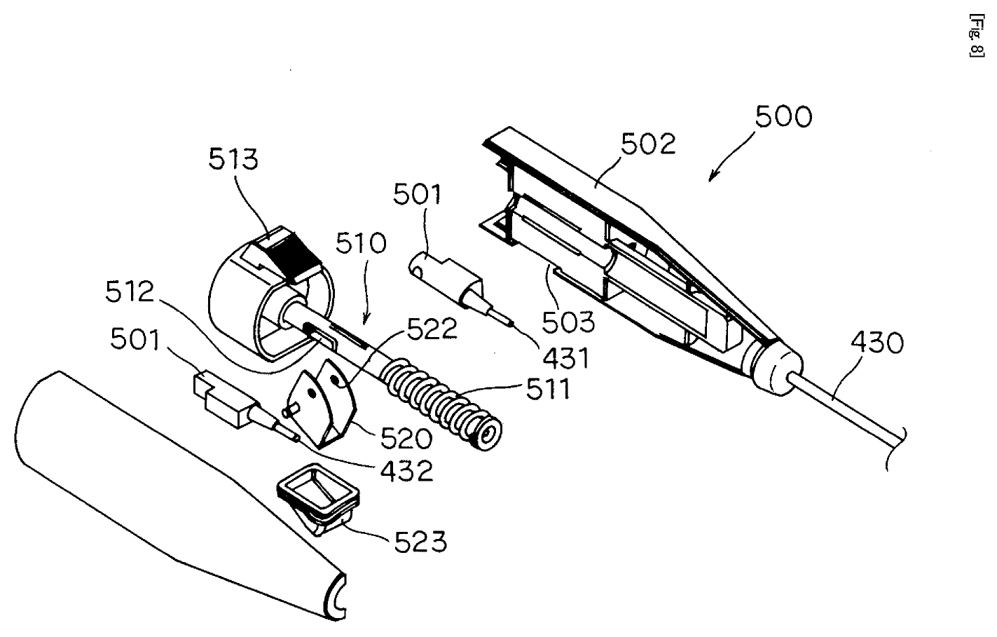

In the above embodiment, the light ray emitted from light-emitting element 401 is always detected by light-receiving element 402. It is possible to provide controller body 420 with a mechanism for starting and stopping the detection through manual operation. A controller unit of such a controller will hereinafter be described in brief with reference to FIGS. 7 to 9. Although the directions of forward, rearward, left, right, up, and down are specified as shown in the description of the present invention, these directions are defined for convenience to simply describe the relative relationship between components and the definition does not limit any direction in manufacture or actual use when the present invention is implemented.

Controller unit 500 shown in the example is connected to fiber cable 430. The trailing ends of first optical fiber 431 and second optical fiber 432 are connected to optical elements 501 for bending the optical axis perpendicularly. Optical elements 501 are fixed to the left and right within controller body 502 and optically coupled to each other.

Shaft member 510 is placed slidably in the forward-and-rearward direction at the center of controller body 502. Shaft member 510 is biased rearward by coil spring 511. Long hole 511 elongated in the forward-and-rearward direction is formed through shaft member 510 in the left-and-right direction, and light-transmitting plate 421 is placed in long hole 511.

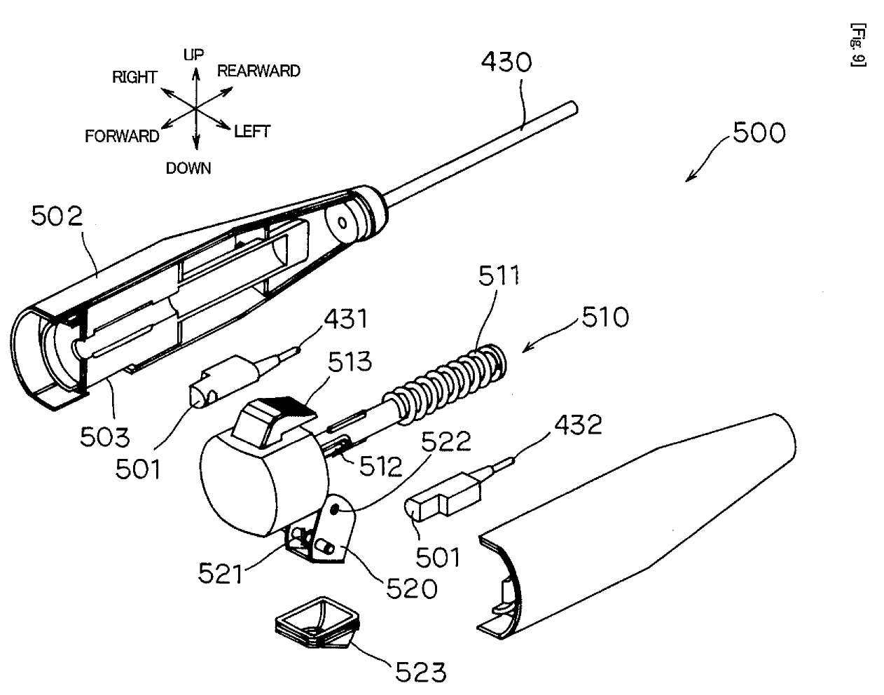

Thus, optical elements 501 at the trailing ends of first and second optical fibers 431 and 432 are optically coupled to each other via light-transmitting plate 421 in shaft member 510. Manual operation member 512 is mounted on the front end of shaft member 510 and is exposed at the front end of controller body 502. As shown in FIG. 9, the front end of controller body 502 is formed in a double-cylindrical shape, and as shown in FIG. 7, the cylindrical rear end of manual operation member 512 is slidably placed in the space between the cylinder-shaped portions of controller body 502.

Shutter member 520 serving as an optical path open/close mechanism is supported swingably in the vertical direction in a lower portion of controller body 502. Shutter member 520 is biased downward by coil spring 521. Through-hole 522 is formed in shutter member 520. When shutter member 521 is located downward by coil spring 521, shutter member 521 blocks the optical coupling between first and second optical fibers 431 and 432. When shutter member 521 is located upward through manual operation, first and second optical fibers 431 and 432 are optically coupled to each other via through-hole 522 of shutter member 520.

Opening hole 503 is formed in a lower portion of controller body 502, and shutter member 520 is exposed at the lower portion from opening hole 503, Flexible cover member 523 made of resin with no light transmittance is mounted at opening hole 503 of controller body 502 and covers shutter member 520.

In controller unit 500 as described above, when shutter member 521 is not manually operated, the optical coupling between first and second optical fibers 431 and 432 is blocked. Only when shutter member 521 is manually operated, first and second optical fibers 431 and 432 are optically coupled.

Thus, an operator who intends to use controller unit 500 moves manual operation member 513 forward to a desired position with his thumb and manually operates shutter member 521 upward with his forefinger, for example. Then, light-receiving element 402 of chemical liquid injector 100 detects a corresponding light ray position of moved manual operation member 513, so that the detection result is held as data to perform the operation accordingly.

When the operator releases the manual operation of shutter member 521, shutter member 521 is lowered to the initial position by the biasing of coil spring 521 to block the optical coupling between first and second optical fibers 431 and 432. Chemical liquid injector 100 continues the operation in accordance with the detection result held as data.

At this point, when the operator releases the manual operation of manual operation member 513, light-transmitting plate 421 is moved rearward to the initial position by the biasing of coil spring 511 together with shaft member 510. Since the optical coupling between first and second optical fibers 431 and 432 is blocked, the operation of chemical liquid injector 100 is not changed.

As described above, in controller unit 500, when shutter member 621 is manually operated for a short time with manual operation member 513 manually operated to the desired position, the desired operation state can be supplied as data to chemical liquid injector 100. Thus, the operator does not need to maintain the operation state of controller unit 500, and the desired operation state can be supplied as data to chemical liquid injector 100 in desired timing.

By manually operating manual operation member 513 with shutter member 521 manually operated, the continuously changing operation state can be supplied as data to chemical liquid injector 100.

In controller unit 500 as described above, manual operation member 513 for moving light-transmitting plate 421 is exposed to the outside from unit body 501. As shown in FIG. 9, the front end of controller body 502 is formed in the double-cylindrical shape, and as shown in FIG. 7, the cylindrical rear end of manual operation member 512 is slidably placed in the space between the cylinder-shaped portions. Thus, outside light can be favorably prevented from entering second optical fiber 432.

In controller unit 500 described above, shutter member 521 is placed at opening hole 503 of unit body 501. Since opening hole 503 is covered with flexible cover member 523 made of resin with no light transmittance, shutter member 521 can be freely operated manually, and outside light can be favorably prevented from entering second optical fiber 432.

1. A controller for controlling operation of a movable mechanism mounted on a body of a movable apparatus, comprising:
a controller body formed separately from the body of the apparatus;
a flexible optical fiber having one end attached to the controller body and the other end attached to the body of the apparatus;
a light-emitting element mounted on the controller body and emitting a light ray which enters the one end of the optical fiber;
a wavelength-variable mechanism mounted on the controller body and changing a wavelength of the light ray emitted by the light-emitting element and entering the one end of the optical fiber in accordance with manual operation;
a light-receiving element mounted on the body of the apparatus and detecting the wavelength of the light ray emitted from the other end of the optical fiber; and
driving control means mounted on the body of the apparatus for controlling operation of the movable mechanism in accordance with the detection result of the light-receiving element.

2. The controller according to claim 1, wherein a controller unit including the controller body, the optical fiber, the light-emitting element, and the wavelength-variable mechanism is formed separately from the body of the apparatus, and the controller unit is removably mounted on the body of the apparatus.

3. The controller according to claim 1, further comprising an optical path open/close mechanism for opening and closing an optical path from the light-emitting element to the one end of the optical fiber in accordance with manual operation.

4. The controller according to claim 1, wherein the wavelength-variable mechanism includes:
a wavelength-variable element for changing a wavelength of a light ray entering from the light-emitting element and emitting to the optical fiber depending on a portion of the wavelength-variable element; and a manual operation member supported movably and operated manually to move at least one of the light-emitting element, the wavelength-variable element, and the one end of the optical fiber.

5. A controller for controlling operation of a movable mechanism mounted on a body of a movable apparatus, comprising:
a controller body formed separately from the body of the apparatus;
a first flexible optical fiber having one end attached to the body of the apparatus and the other end attached to the controller body;
a light-emitting element mounted on the body of the apparatus and emitting a light ray which enters the one end of the first optical fiber;
a second flexible optical fiber having one end attached to the controller body, the light ray emitted from the other end of the first optical fiber entering the one end, and the other end attached to the body of the apparatus;
a wavelength-variable mechanism mounted on the controller body and changing a wavelength of the light ray emitted from the other end of the first optical fiber and entering the one end of the second optical fiber in accordance with manual operation;
a light-receiving element mounted on the body of the apparatus and detecting the wavelength of the light ray emitted from the other end of the second optical fiber; and
driving control means mounted on the body of the apparatus for controlling operation of the movable mechanism in accordance with the detection result of the light-receiving element.

6. The controller according to claim 5, wherein a controller unit including the controller body, the first optical fiber, the wavelength-variable mechanism is formed separately from the body of the apparatus, and the controller unit is removably mounted on the body of the apparatus.

7. The controller according to claim 5, further comprising an optical path open/close mechanism for opening and closing an optical path from the other end of the first optical fiber to the one end of the second optical fiber in accordance with manual operation.

8. The controller according to claim 5, wherein the wavelength-variable mechanism includes:
a wavelength-variable element for changing a wavelength of a light ray entering from the first optical fiber and emitting to the second optical fiber depending on a portion of the wavelength-variable element; and a manual operation member supported movably and operated manually to move at least one of the other end of the first optical fiber and the one end of the second optical fiber.

9. The controller according to claim 4, wherein the wavelength-variable element comprises a light-transmitting plate which transmits light rays with different wavelengths in its different portions.

10. The controller according to claim 4, wherein the wavelength-variable element comprises a reflective plate which reflects light rays with different wavelengths in its different portions.

11. The controller according to claim 4, wherein the wavelength-variable element comprises a prism.

12. The controller according to claim 1, further comprising:
an automatic returning mechanism for returning the wavelength-variable mechanism to an initial state; and
an operation holding mechanism for releasably holding the wavelength-variable mechanism in an operation state.

13. The controller unit of the controller according to claim 2, comprising:
the controller body formed separately from the body of the apparatus;
the flexible optical fiber having one end attached to the controller body and the other end removably attached to the body of the apparatus; and
the wavelength-variable mechanism mounted on the controller body and changing a wavelength of the light ray emitted by the light-emitting element and entering the one end of the optical fiber in accordance with manual operation.

14. The controller unit of the controller according to claim 6, comprising:
the controller body formed separately from the body of the apparatus;
the flexible first optical fiber having one end removably attached to the body of the apparatus and the other end attached to the controller body;
the flexible second optical fiber having one end attached to the controller body, the light ray emitted from the other end of the first optical fiber entering the one end, and the other end removably attached to the body of the apparatus; and
the wavelength-variable mechanism mounted on the controller body and changing a wavelength of the light ray emitted from the other end of the first optical fiber and entering the one end of the second optical fiber in accordance with manual operation.

15. The movable apparatus on which the controller unit according to claim 13 is removably mounted, comprising:
the body of the apparatus;
the movable mechanism mounted on the body of the apparatus;
a light-receiving element mounted on the body of the apparatus and detecting a wavelength of the light ray emitted from the other end of the optical fiber;
a driving control means mounted on the body of the apparatus for controlling operation of the movable mechanism in accordance with the detection result of the light-receiving element.

16. The movable apparatus on which the controller unit according to claim 14 is removably mounted, comprising:
the body of the apparatus;
the movable mechanism mounted on the body of the apparatus;
a light-emitting element mounted on the body of the apparatus and emitting a light ray which enters the controller unit;
a light-receiving element mounted on the body of the apparatus and detecting a wavelength of the light ray emitted from the controller unit;
a driving control means mounted on the body of the apparatus for controlling operation of the movable mechanism in accordance with the detection result of the light-receiving element.

17. The movable apparatus according to claim 15, wherein the movable mechanism individually holds a cylinder member and a piston member of a liquid syringe, the piston member being slidably inserted into the cylinder member, such that the piston member is moved relatively to the cylinder member.

18. The controller according to claim 8, wherein the wavelength-variable element comprises a light-transmitting plate which transmits light rays with different wavelengths in its different portions.

19. The controller according to claim 8, wherein the wavelength-variable element comprises a reflective plate which reflects light rays with different wavelengths in its portions.

20. The controller according to claim 8, wherein the wavelength-variable element comprises a prism.

21. The controller according to claim 5, further comprising:
an automatic returning mechanism for returning the wavelength-variable mechanism to an initial state; and
an operation holding mechanism for releasably holding the wavelength-variable mechanism in an operation state.

22. The movable apparatus according to claim 16, wherein the movable mechanism individually holds a cylinder member and a piston member of a liquid syringe, the piston member being slidably inserted into the cylinder member, such that the piston member is moved relatively to the cylinder member.
